(19) United States
(12) Patent Application Publication
(10) Pub. No.: US 2023/0181079 A1
(43) Pub. Date: Jun. 15, 2023

(54) BODY ELECTRODE FOR RECORDING ELECTRO-PHYSIOLOGICAL SIGNALS

(71) Applicant: Piotrode Medical AB, Saltsjö-Boo (SE)

(72) Inventors: Krister SJÖBERG, Kungsängen (SE); Göran SJÖBERG, Kungsängen (SE); Per ASK, Linköping (SE)

(73) Assignee: Piotrode Medical AB, Saltsjö-Boo (SE)

(21) Appl. No.: 17/911,981

(22) PCT Filed: Mar. 5, 2021
(86) PCT No.: PCT/SE2021/050197

§ 371 Date: Sep. 15, 2022

(30) Foreign Application Priority Data
Mar. 16, 2020 (SE) 2050287-8

(51) Int. Cl. A61B 5/25; A61B 5/30

(52) U.S. Cl. CPC A61B 5/25; A61B 5/30; A61B 5/28

(57) ABSTRACT

The present invention relates to a body electrode for recording electrophysiological signals from a body. In particular the invention relates to a body electrode (100; 200; 400) comprising a transducer element (105; 205) shielded by a layered shield structure (120; 220; 420) and a skin contact element (115; 115′; 115″; 215) providing a contactbetween the layered shield structure (120; 220; 420) and the skin (101; 201) of the body. The layered shield structure (120; 220; 420) comprises at least an electrically conducting layer (113; 213) and an electrostatic dissipative layer (112; 212; 412). The skin contact element (115; 115′; 115″; 215) comprises an electrically onducting layer (113; 213) and an ion conducting layer (114; 214) and is with regards to the electrical potential characteristics matched with a transducer element (105; 205).

### FIELD OF INVENTION

The present invention relates to a body electrode for recording electrophysiological signals from a body. In particular the invention relates to a shielded body electrode providing a contact between the shield and the skin of the body.

### BACKGROUND OF THE INVENTION

Electrodes applied on the skin surface of a subject, e.g. a human, can be used to record electrophysiological signals produced e.g. by the heart, i.e. an electrocardiogram (ECG), by the brain, i.e. an electroencephalogram (EEG), by the eyes, i.e. an electroretinogram (ERG) and/or an electrooculogram (EOG). The quality of such a recording is limited by the performance of the used electrodes. The electrodes may be subject for different disturbances that in turn give rise to disturbances in the output of the recorded electrophysiological signals. One such disturbance is caused by electrostatic fields surrounding the body electrode. The electrostatic fields may give rise to electrostatic induction which may cause disturbances. Such disturbances may create signal amplitudes that can be many times the size of the electrophysiological signal to be recorded, and hence may deteriorate accurate recording of electrophysiological signals. Electrostatic fields can be generated by e.g. clothes, electrode cable movements, objects in the surrounding etc. The signals from electrostatic induced disturbances has a frequency content overlapping the electrophysiological signals which makes it difficult to use conventional soft—and hardware filters to remove it from the recording.

US7993167B2 discloses an ECG lead set which is shielded against electrostatic disturbances by an electrical shield. The electrical shield is covered by a nonconductive cover and is electrically connected to the shield of the coaxial cable of the lead set.

JP2013022150A discloses an electrostatic induction noise suppressor device for a bioelectrode, and a method for suppressing electrostatic induction noise in a biological signal detected by a biological electrode. The electrostatic induction noise suppressing device has a discharge part for discharging a charging charge of the charging part to the living body via a connecting part for electrically connecting the charging part and the body surface.

In the prior art there is a need for an improved electrostatic protection of a body electrode, and to improve the quality of a recording of electrophysiological signals from body electrodes by minimizing the influence caused by electrostatic fields.

### SUMMARY

The object of the invention is to provide a body electrode that overcomes at least some of the drawbacks of prior art. This is achieved by the body electrode as defined in claim 1, the body electrode arrangement as defined in claim 17 and the measurement system as defined in claim 18.

According to one aspect of the invention a body electrode for electrophysiological signal monitoring is provide. The body electrode is during used arranged to be attached to the skin of a subject, and comprises:
a collar with at least one through opening;
a transducer element at least partly arranged within the through-opening of the collar;
a skin facing surface arranged to, during use, be in contact with the skin and a free surface opposite of the skin facing surface;
a connector in electrical contact with the transducer element, the connector arranged on the free surface; and
an electrically conducting compartment formed by the wall of the through-opening of the collar and comprising the transducer element, the electrically conducting compartment during use comprising an electrolyte medium. The body electrode further comprises a layered shield structure arranged on the free surface of the body electrode. The layered shield structure comprises at least an electrically conducting layer and an electrostatic dissipative layer arranged on the surface of the electrical shield layer and wherein the layered shield structure is covering at least the electrically conducting compartment, the connector and the transducer element. The body electrode comprises a skin contact element in electrical contact with the layered shield structure and the skin contact element comprises at least an ion conducting layer and a layer of an electrically conducting material. The skin contact element is arranged to during use be in contact with the skin. The materials of the layers of the skin contact element are selected so that the electrical potential of the skin contact element matches the electrical potential of transducer element.

According to one embodiment of the invention the skin contact element is formed from the layered shield structure and the ion conducting layer is a continuous layer including the skin contact element.

According to one embodiment the layered shield structure of the body electrode further comprises an ion-conducting layer, and the ion-conducting layer of the skin contact element is arranged to be connected with the ion-conducting layer of the layered shield structure.

According to one embodiment of the invention the layered shield structure comprises an ionic conducting layer arranged below the electrically conducting shield and the layered shield structure covers at least the electrically conducting compartment, the connector and the transducer element.

According to one embodiment of the invention the skin contact element is a wire-like structure extending from the electrical shield layer on the outer peripheral of the collar and over at least a portion of the skin facing surface of the body electrode.

According to one embodiment of the invention the layered shield structure further comprises at least one flap arranged to during use spatially overlap with a lead shield.

According to one embodiment of the invention at least one of the materials in the layers of the skin contact element differs from the materials in the layers of the transducer element.

According to one embodiment of the invention the potential difference U_{diff}, between the transducer element and the electrically conducting layer is lower than 30 mV.

According to embodiments of the invention the electrically conducting layers of the body electrode and/or the skin contact element is provided as a layer comprising an ion carrier material comprising an ion solution or to which an ion solution may be provided before use. Thereby the electrically conducting layer(s) functions as both the electrically conducting layer and the ion conducting layer.

According to one embodiment of the invention the ion conducting layer comprises a polymer matrix and a water-soluble salt. The polymer matrix may comprise a hydrogel.

According to one embodiment of the invention the electrically conducting layer comprises a carbon-based polymer material and the ion conducting layer comprises an acrylic material.

According to one embodiment of the invention the ion conducting layer is adhesive. The ion conducting layer may be provided as an adhesive layer that is arranged to, during use, also fastens the skin contact element to the skin.

According embodiments of the invention the transducer element comprises Ag/AgCl. A matching skin contact may be provided by
a skin contact element comprising Ag and a gel or hydrogel with at least 0.6% by mass of a chloride salt,
a skin contact element comprising Ag coated with AgCl and a gel or hydrogel with at least 0.6% by mass of a chloride salt,
a skin contact element comprising carbon coated with an acrylic polymer matrix, and wherein the polymer matrix forms both the electrically conducting layer and the ion conductive layer.

There is an advantage with the invention that the potential difference, as given by the U_{diff} value can be reduced and/or stabilized. A reduced and/or stabilized U_{diff} value may result in reduced disturbances during an electrophysiological measurement. To match the electrical potential characteristics of the transducer element and the skin contact element by the design of the body electrode has advantages over applying filtering or signal processing at a later stage since the disturbances otherwise caused may be similar to the signal variations to be detected.

A further advantage with the invention is that there is no openings/holes present that may allow electrostatic fields to penetrate through the shield structure and reach the conducting compartment where it may cause disturbances.

According to one aspect of the invention a body electrode arrangement is provided comprising least one body electrode an indifferent body electrode and a hub device. Each of the body electrodes comprises a transducer element that during use is connected to a measuring device via a signal conductor. In the body electrode each of the body electrodes comprises a layered shield structure comprising an electrically conducting layer and an electrically dissipative layer. An isolated lead is arranged to pass the hub device and electrically connect the electrically conducting layers of the each of the body electrodes and the indifferent body electrode provides a skin contact element that is arranged to be in electrical contact with the isolated lead.

According to one embodiment the indifferent body electrode comprises a transducer element utilized as a skin contact element and the isolated lead is connected to the transducer element of the indifferent electrode.

According to one embodiment the indifferent electrode is a body electrode comprising a skin contact element as described above, and wherein the skin contact element of the indifferent electrode is connected to the isolated lead.

There is an advantage with the invention that one body electrode function as a skin contact element. Then the other body electrodes do not need to comprise a skin contact element.

According to one aspect of the invention a measurement system is provided comprising a body electrode according to the above aspects of the invention. The measurement system comprises means configured to:
measure an electric potential of the transducer element; and
provide an electric potential to the electrically conducting shield layer, wherein the electric potential is based on the measured electric potential in step, so that the potential difference, U_{diff}, between the layered shield structure; and the transducer element is lower than 30 mV, preferably lower than 15 mV, and even more preferably lower than 10 mV.

According to one aspect of the invention a measurement system is provided connected to the body electrode arrangement described above and further comprises means configured to:
measure an electric potential via the indifferent electrode; and
provide the connector shields of the body electrodes with an electric potential, based on the measured electric potential from the indifferent electrode wherein the electric potential is based on the measured electric potential, so that the potential difference, U_{diff}, between the layered shield structure and the transducer element is lower than 30 mV, preferably lower than 15 mV, and even more preferably lower than 10 mV.

### DESCRIPTION OF DRAWINGS

FIG. 1a-e shows schematic illustrations of a body electrode according to the invention, wherein FIG. 1a shows an exploded view of a schematic illustration of a body electrode according to the invention, FIG 1b-d shows schematic illustrations of a body electrode according to the invention, FIG. 1e shows an exploded view of a schematic illustration of a body electrode according to the invention;

FIG. 2 shows a schematic illustration of a body electrode according to the invention;

FIG. 3a and b shows a schematic illustration of one embodiment of the invention;

FIG. 4a, b and c shows schematic illustrations of one embodiment of to the invention;

FIG. 5 shows schematic illustrations of a body electrode according to the invention;

FIG. 6 shows a schematic illustration of electric potentials;

FIG. 7 shows schematic illustration of a measurement test equipment;

FIG. 8 shows a schematic illustration of one embodiment of the invention; and

FIG. 9 shows electrocardiograms recorded using body electrodes.

### DETAILED DESCRIPTION

Terms such as “top”, “on top”, “bottom”, upper“, lower”, “below”, “above” etc. are used merely with reference to the geometry of the embodiment of the invention shown in the drawings and/or during normal operation of the described device and system and are not intended to limit the invention in any manner. The aim of a body electrode is to receive and record electrophysiological signals from a body. The signals are recorded by a transducer element and transmitted to various medical instruments. Such recordings may be disturbed by electrostatic induction surrounding the body electrode. Electrostatic induction may be caused by changing electrostatic fields surrounding the body electrode. Such electrostatic fields can for example be generated by clothing or other covers, electrode cable movements, the surrounding environment etc. Disturbances at the interface between the electrolyte medium and the skin may cause changes of the potential at the interface which may result in disturbances in the recorded electrocardiogram, wherein “potential” refers to an electric potential and/or an electrochemical potential, typically measurable with a common voltmeter and/or oscilloscope. Disturbances may be reduced and/or stabilized using a body electrode according to the invention.

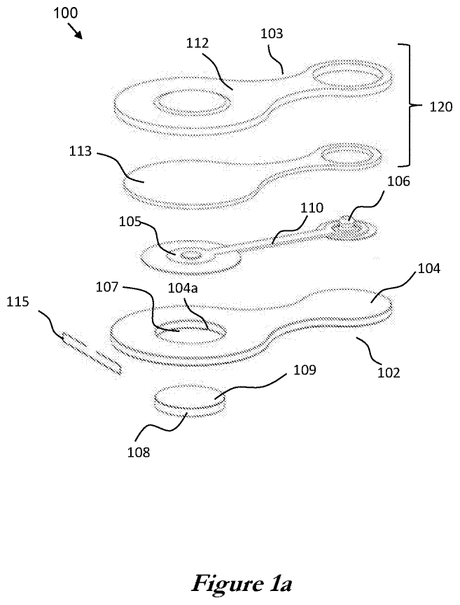

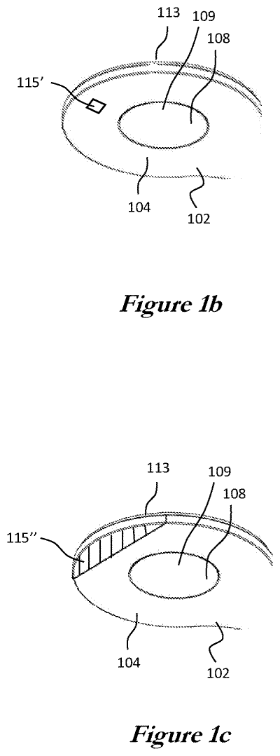

A body electrode according to the invention is schematically illustrated in FIGS. 1a-e, that shows a schematic illustration of a body electrode 100 in exploded view. The body electrode 100 has two surfaces: a skin facing surface 102 arranged to be in contact with the skin during use of the body electrode 100, and an opposite free surface 103. The body electrode 100 comprises a collar 104 having at least one through-opening 107. The collar 104 is arranged in between the skin facing surface 102 and the free surface 103. Alternatively, the upper side of the collar 104 forms the free surface 103, or part of it, and/or the lower side of the collar 104 forms the skin facing surface 102, or part of it. A transducer element 105 is arranged, at least partly, in the through-opening of the collar 107, and a connector 106 is arranged in electrical contact with the transducer element 105. The connector 106 is arranged to be accessible on the free surface 103 and to receive a matching lead connector. The through-opening 107 defines a conductive compartment 108 which in the direction parallel to the skin facing surface 102 is limited by the wall of the collar 104a. The conductive compartment 108 comprises an electrolyte medium 109, at least during use of the body electrode 100. The electrolyte medium 109 allows the transducer element 105 to be in electrical contact with the skin during use of the body electrode 100. The electrolyte medium 109 may be a liquid, a liquid gel, a hydrogel (solid), sweat, etc.

The body electrode 100 further comprises a layered shield structure 120 arranged so that it forms at least part of the free surface 103. The layered shield structure 120 comprises an electrostatic dissipative layer 112 and an electrically conducting layer 113, with the dissipative layer 112 on top of the electrically conducting layer 113 as seen from the collar 104.

According to one embodiment the layered shield structure 120 is arranged so that it covers at least the transducer element 106, the connector 106, the lead 110 and the conductive compartment 108.

In one embodiment the connector 106 is arranged on top of the transducer element 105, in such case there may be no lead 110 and the layered shield structure 120 may be arranged so that it covers at least the connector 106, the transducer element 105, and the conductive compartment 108.

In an embodiment illustrated in FIG. 1d and 1e, the connector 106 is not covered by the layered shield structure 120, the layered shield structure 120 comprises a through opening 120′ in through which the connector 106 is accessible. To provide for an effective shielding the connector 106 is arranged to mate with a corresponding lead connector 106′. The lead connector 106′ is provided with layered shield structure 120′, comprising at least an electrically conducting layer and an electrostatic dissipative layer. The body electrode 100 and the lead connector 106′ are arranged so that in the mounted position the electrically conducting layer 113 of the body electrode 100 is in electrical contact with the electrically conducting layer of the lead connector 106′. The electrostatic dissipative layer 112 of the body electrode is arranged to be in physical contact with the dissipative layer of the lead connector 106′. In this way the layered shield structure 120 and the layered shield structure of the lead connector 106′ is electrically connected to the each other forming a unified electrical shield structure. The lead connector 106′ is connected to a signal recording device (not shown) via a lead that may comprise a lead shield 111, in such case the layered shield structure of the lead connector 106′ may spatially overlap with the lead shield 106′. The layered shield structure of the lead connector 106′ may be arranged so that it, or one or more of its layers, extends a radial distance out over the layered shield structure 120 of the body electrode 100 forming overlapping shield structures.

According to one embodiment an aggregate 130 comprising a body electrode 100 and a lead connector 106′ comprising a layered shield structure is provided, wherein the layered shield structures of the lead connector 106′ and the body electrode 100 forms a unified electrical shield structure.

In an embodiment the lead connector 106′ may be a clip-on, or clamp, connector type that is arranged to be clamped onto the connector 106 of the body electrode 100. Such connector types are known in the art. The layered shield structure of such a clip-on connector would typically be in the form of two parts that during mounting would open up to receive the connector 106. After mounting, and the two parts of the layered shield structure is closed and electrically connected again forming a unified connector shield without any holes or openings. Alternatively, the two parts of the layered shield structure of the lead connector 106′ are arranged to overlap in the closed position.

In one embodiment the connector 106 is arranged at a distance from transducer element 105 and connected with an electrical lead 110.

The body electrode 100 further comprises a skin contact element 115 that at least during use of the body electrode 100 comprises an ion conducting layer 114′ and an electrically conducting layer 113′ as schematically illustrated in the in the enlarged view of Figure le. The skin contact element 115 may further, according to embodiments of the invention, be provided with an electrostatic dissipative layer 112′. The layered structure is relevant for all variants of the skin contact element 115, for example the embodiments described with reference to FIGS. 1a (skin contact element 115), 1b an 1e (skin contact element 115′), and 1c (skin contact element 115″).

According to one embodiment depicted in FIG. 1a, the skin contact element 115 is provided as a wire-like structure that is connected to the electrical shield layer 113 and extends to the skin facing surface 102 on the outer peripheral of the collar 104. According to one embodiment the wire-like structure extends from the electrical shield layer 113 on the outer peripheral of the collar 104 and over the skin facing surface 102 of the body electrode 100.

In an alternative embodiment, schematically illustrated in FIG. 1b, the skin contact element 115′ is provided on the skin facing surface 102 and extends through a through-hole in the collar 104 and possibly through other layers and connects with the electrically conducting layer 113. An exploded view of such an embodiment is schematically illustrated in FIG. 1e. In FIG. 1e, the skin contact element 115′ is provided on the skin facing surface 102 and extends through a through-hole in the collar 104 and possibly through other layers and connects with the electrically conducting layer 113. The skin contact element 115″ comprises at least an ion conducting layer 114′ and an electrical layer 113′.

In yet another embodiment, schematically illustrated in FIG. 1c, the collar 104 is provided with a cut-out in which the skin contact element 115″ is provided and forms a portion of the skin facing surface 102. The skin contact element 115″ extends to, or is arranged to be in electrical contact with, the electrically conducting layer 113.

The skin contact element 115; 115′; 115″ is in contact with the skin during use of the body electrode 100. The skin contact element 115; 115′; 115″ is in electrical contact with the electrically conducting layer 114. In that way, the skin contact element 115; 115′; 115″ enables charge transfer between the layered shield structure 120 and the skin during use of the body electrode 100.

The materials and the thickness of the layers, i.e. the electrically conducting layer 114′ and the ion conducting layer of the skin contact element 115; 115′; 115″ are selected so that the electrical potential of the skin contact element 115; 115′; 115″ matches the electrical potential of the transducer element 105 comprised in the conductive compartment 108. That the electrical potentials are matching should herein be understood as that the potential difference, U_{diff}, between the electrical shield layer 113 and the transducer element 105 is kept lower than 30 mV, or lower than 20 mV, or lower than 15 mV, or lower than 10 mV, or lower than 7 mV, or lower than 5 mV during use of the body electrode.

The layered shield structure 120 comprising the electrically conducting layer 113 and the electrostatic layer 112 may be arranged at a distance from the transducer element 105 so that the transducer element 105 and the layered shield structure 120 are not in contact at least during use of the body electrode 100. Furthermore, the layered shield structure 120 may be comprehensive so that it does not comprise any openings/holes larger than 1.5×0.5 mm.

The electrostatic dissipative layer 112 is arranged on top of the body electrode 100 forming at least part of the free surface 103. The electrically dissipative layer 112 may for example comprise a polymer, an elastomer, a woven or non-woven textile or a mixture thereof. The electrostatic dissipative layered shield structure 112 preferably have a surface resistivity of 10⁵-10¹¹ Ohms per square.

The electrically conducting layer 113 is arranged underneath the electrostatic dissipative layer 112. The electrically conducting layer 113 may have a surface resistivity that is equal or less than 10⁻¹-10³ Ohms per square. The electrically conducting layer 113 may comprise: a metal, an electrically conducting carbon paint, a carbon-based polymer, an electrically conducting polymer or an ionic polymer. The electrically conducting layer 113 may alternatively comprise a material, referred to as an ion carrier, that can be loaded with an ion solution for example a woven or non-woven material that comprises an ion solution e.g. a liquid containing water-soluble salts, or a mixture thereof. The electrostatic dissipative layer 112 and the electrically conducting shield layer 113 are in contact with each other so that charge can drain from the electrostatic dissipative layer 112 to the electrically conducting shield layer 113.

The skin contact element 115; 115′; 115″ comprises an ion conducting layer 114′ and an electrically conducting layer 113′ and is in electrical contact with the layered shield structure 120. The ion conducting layer 114′ may for example be a liquid gel and a water-soluble salt or a hydrogel with a water-soluble salt, or a polymer matrix and a water-soluble salt or a polymer matrix that is both ion-conducting and electrical conducting. A water-soluble salt should be understood as a salt having an aqueous solubility of at least 100 g/1000 ml water at 25° C., e.g. NaCl, KCl and CaCl₂. with concentrations of for example 3 g salt per 97 g of water equal to 3% by mass. The ion conducting layer may also comprise an acrylic material.

The transducer element 105 may for example be a silver/sliver chloride type.

The body electrode 100 may for example be in the form of a square with a side of 25-55 mm and having a collar of 0.5-3 mm in thickness. Or the body electrode 100 may have the shape of a circle with a diameter of 25-55 mm and a collar of 0.5-3 mm in thickness. For such body electrodes the layered shield structure may be in the form of a circle having a diameter of 15-25 mm or in the shape of a square having a side of 15-25 mm

For such body electrodes as described above the area of the skin contact element 115, 115′, 115″ or 215 may be between 4 mm² and 100 mm²

The skin contact element 115 may additionally be in the form of single strand wire for example comprising silver, the single strand wire may have a surface contacting part being 10-30 mm long and 0.1-1 mm in diameter.

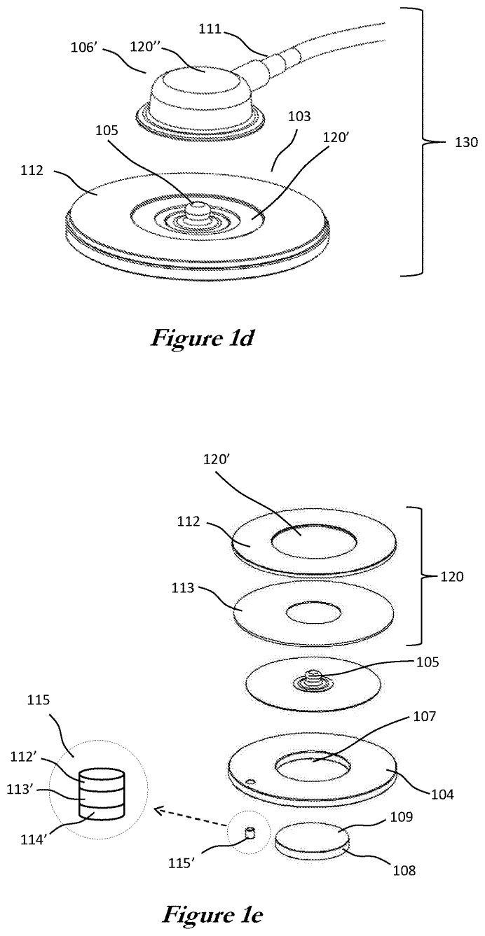

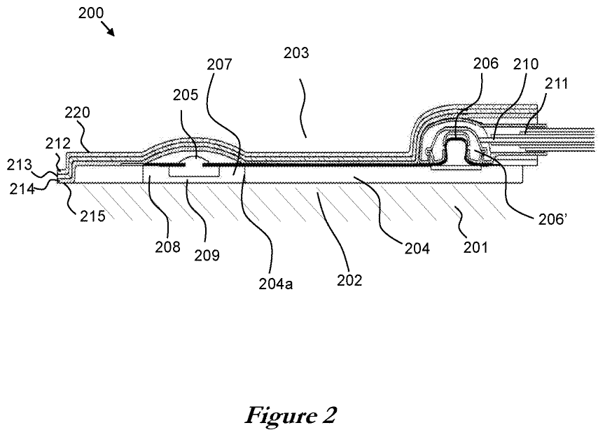

In one embodiment of the invention, schematically illustrated in FIG. 2 the skin contact element 215 is formed from the layered shield structure 220. FIG. 2 shows a schematic illustration of a body electrode 200 in cross-section. The body electrode 200 is arranged to be attached to the skin 201 of a subject, e.g. a human or an animal. The body electrode 200 has two surfaces: a skin facing surface 202 arranged to be in contact with the skin 201 and an opposite free surface 203. The body electrode 200 comprises a collar 204 having at least one through-opening 207. The collar 204 is arranged in between the skin facing surface 202 and the free surface 203. Alternatively, the upper side of the collar 204 forms the free surface 203, or part of it, and/or the lower side of the collar 204 forms the skin facing surface 202, or part of it. A transducer element 205 is arranged, at least partly, in the through-opening of the collar 207, and a connector is 206 arranged at a distance from the transducer element 205, the connector 206 is in electrical contact with the transducer element 205. The connector 206 is connected to a signaling recording device (not shown) via a mating lead connector 206′ in connection with a lead 210. The lead 210 is covered by a lead shield 211, the layered shield structure 220 may overlap with the lead shield 211, so that at least partly covers the connector 206 and the lead shield 211 and/or the lead 210. In such case if the layered shield structure 220 overlaps spatially with the lead shield 211 there may be no electrical contact between the layered shield structure 220 and the lead shield 211. The lead connector 206′ may have a connector shield 206″ that also extends over the lead shield 211.

The body electrode 200 further comprises a conductive compartment 208 which in the direction parallel to the skin facing surface 202 is limited by the wall 204a of the collar 204, the conductive compartment 208 comprises an electrolyte medium 209 at least during use of the body electrode 200. The body electrode 200 further comprises a layered shield structure 220 arranged on the free surface 203 so that it forms at least part of the free surface 203. The layered shield structure 220 comprises an electrostatic dissipative layer 212, an electrically conducting layer 213, and an ion conducting layer 214. The dissipative layer 212 is arranged on top off the electrically conducting layer 213 on the side that faces away from the free surface 203. The ion conducting layer 214 is arranged in contact with the electrically conducting layer 213 on the side that faces away from the free surface 203. The ion conducting layer 214 is arranged to be in contact with the skin 201 via a skin contact element 215 formed by the layered shield structure 220.

According to one embodiment illustrated in FIG. 2 the skin contact element 215 is formed by the layered shield structure 220 extending over and around the edge of the collar 204, is extended in the direction towards the skin facing surface 202 and is folded so that the ion conducting layer 213 is a continuation of the skin facing surface 202 of the collar 204. The skin contact element 215 may extend all the way around the body electrode's 100 circumference or, alternatively, extend over a portion of the circumference. The three-layer structure 220 may for example be in the form of a tape. Such tape may be applied on a body electrode 200, covering at least the connector 206, the transducer element 205, and the conducting compartment 208. The tape may further be applied so that it overlaps spatially with the lead shield 211 when the connector 206 is connected to a signaling recording device via a lead 210 covered with a lead shield 211. Suitable dimensions of the skin contact element 215 are an extension from the collar 104 of 2-100 mm and in the circumferential direction 2-10 mm.

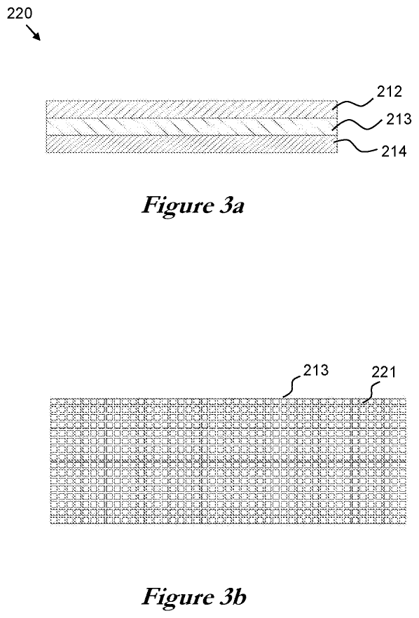

FIG. 3a shows an enlarged view of the layered shield structure 220 according to the embodiment according to the invention. It shows a part of a cross-section of a layered shield structure 220 arranged as a three-layer structure comprising the electrostatic dissipative shield layer 212, the electrically conducting layer 213 and the ion conducting layer 214. The electrically conducting layer 213 is arranged in between the electrostatic dissipative layer 212 and the ion conducting layer 214. FIG. 3b shows a schematic illustration of an embodiment, wherein the electrical shield 313 comprises a grid pattern 221. In an example of a layered shield structure 220 comprising a grid pattern 221 the electrically dissipative layer 212 may have a surface resistivity of 10¹¹ Ohms or below, e.g. comprising a polymeric material such as polypropylene (PP) or polyethene (PE). The electrically conducting layer 213 may be composed of conductive printed ink that is printed in a grid pattern of e.g. 1×1 mm, or 0.5×0.5 mm, on the electrically dissipative layer 212, the printed grid pattern having a surface resistivity of 10³ Ohms or below. The ion conducting layer 214 may be both ion conducting and adhesive, in this way the three-layer structure 220 in the form of a tape may be used to attach the body electrode 200 and/or connector 206 to the skin 201. Such a three-layer structure 220 comprising a grid pattern 221 may be beneficial in terms of material properties such as flexibility and manufacturing properties. Such a three-layer structure 220 may have a total thickness of approximately 40 to 100 micrometer.

The ion conducting layer 214; 314 may comprise an ionic gel, semi-gel or a hydrogel. According to one embodiment the ion conducting layer 214; 314 is in the form of an adhesive. The adhesive ion conducting layer 214; 314 may in this embodiment serve several functions: as the ion conducting material providing the electrical connection to the skin during use, to adhere the skin contact element 215 to the skin and to adhere the electrical shield layer 213 to the underlying parts of the body electrode 200.

The body electrode 100; 200 may also comprise an additional layer that is adhesive and arranged at the skin facing surface 102; 202 to attach the body electrode 100; 200 to the skin 101.

The body electrode 100; 200 may additionally comprise additional layers for example additional shields, e.g. electrical and/or electrostatic dissipative, at least partly surrounding electrical parts of the body electrode 100; 200, such the lead 110; 210, and/or connector 106; 206, etc.

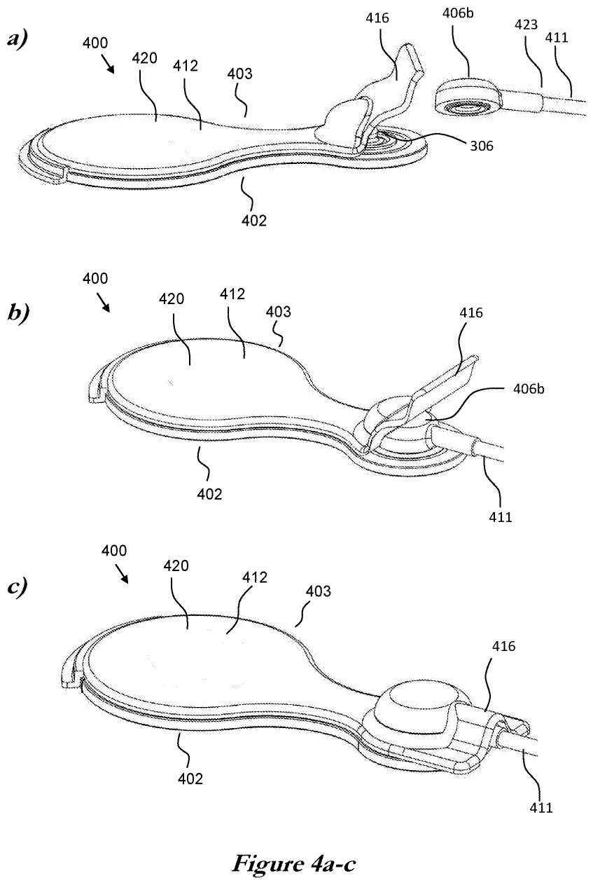

FIG. 4 is a schematic illustration of one embodiment according to the invention. FIGS. 4a, b and c show schematic illustrations of a body electrode 400 in elevated view. The body electrode 400 is via connector 406 during use connected to a mating lead connector 406′, as illustrated in FIGS. 4a-c. The lead connector 406′ is connected to a signal conductor 423. The signal conductor 423 comprises a lead (not shown) provided with a lead shield 411 connected to a signal recording device (not shown). The body electrode 400 is covered by a layered shield structure 420. The layered shield structure 420 comprises at least an electrically conducting layer (not shown) and an electrostatic dissipative layer 412. Optionally, the layered shield structure 420 comprises an ion conducting layer (not shown) arranged closest to the skin facing surface 402 of the layers in the layered shield structure 420. The electrostatic dissipative layer 412 is arranged on top off the electrically conducting layer facing the free surface 403 of the body electrode 400 so that it forms at least parts of the free surface 403. The body electrode 400 further comprises a flap 416, so that part of the layered shield structure 420 constitutes the flap 416. The flap 416 is attached to the body electrode 400 at one edge of the flap 416 so that it is free to move in at least one direction in a folding manner. In other embodiments the flap 416 may be in the form of a triangle, or circle or another shape. The flap 416 is arranged to at least partly cover the connector 406, and during use at least partly cover also the lead connector 406′ and/or the lead and lead shield 411. Preferably the flap 416 is arranged so that the lead connector 406′ and/or the lead and lead shield 411 are covered by the layered shield structure 420 so that the layered shield structure 420 overlaps with the lead shield 411 during use of the body electrode 400, as schematically illustrated in FIG. 4c.

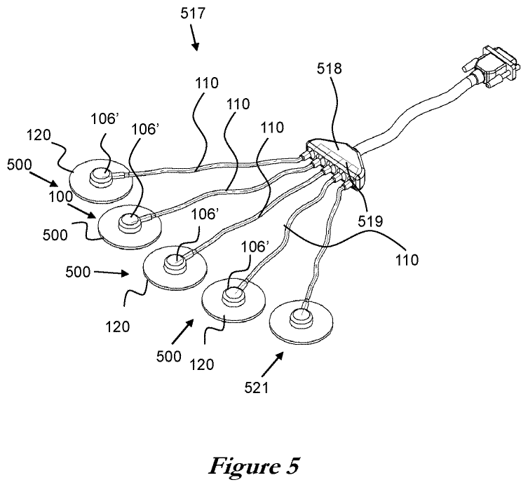

According to one embodiment of the invention there may be an aggregate arrangement 517 comprising several body electrodes 500, such as five body electrodes for example, and a hub device 518. Each body electrode 500 comprises a layered shield structure 120 comprising an electrically conducting layer 113 and an electrically dissipative layer 112, and a lead connector 106′ covered by a layered shield structure. Such an embodiment is schematically illustrated in FIG. 5. Each of the body electrodes 500 comprises a transducer element 105 that is connected to a measuring device (not shown) via a signal conductor 110. The body electrodes 500 are further connected via an isolated lead 519, e.g. a single strand lead, that passes the hub device 518, to each other. The isolated lead 519 provides an electrical connection so that the conducting layers, i.e. the conducting layers 112 of the layered shield structures 120 and the electrical layers of the conducting layer of the layered shield structure of the lead connector 206′ , of the body electrodes 500 are in electrical contact with each other.

According to one embodiment of the aggregate arrangement 517 one of the body electrodes is an indifferent electrode 521, that has a different function and/or design than the other body electrodes. The indifferent electrode 521 is provides a skin contact element for the aggregate arrangement 517.

According to one embodiment the indifferent body electrode 521 comprises a transducer element utilized as a skin contact element and the isolated lead 519 is connected to the transducer element of the indifferent electrode 521.

According to one embodiment the indifferent body electrode 521 the indifferent electrode is a body electrode 100, 200, 400 as described above, with its skin contact element 115; 115′; 115″ connected to the isolated lead 519.

According to one embodiment of the aggregate arrangement 517 at least one of the body electrodes 100 comprises a skin contact element 115 and is used to contact the skin during use of the aggregate arrangement 517. In such an embodiment the skin contact element 115 may be connected to the other body electrodes 100 via the isolated lead 519.

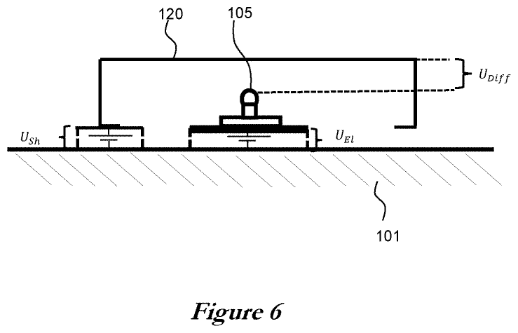

The body electrode 100; 200; 400 will during use exhibit potential differences between different parts, as schematically illustrated by the equivalent circuit of FIG. 6. The equivalent electric circuit associated with the body electrodes described with reference to FIGS. 1 to 4 is illustrated during use or during testing of the body electrodes. FIG. 6 illustrates a potential difference Ush between the layered shield structure 120 and the skin 101, and a potential difference Uₑₗ between the transducer element 105 and the skin 101, and a potential difference Udff between the layered shield structure 120 and the transducer element 105. The relation between the potential differences is:

U_{diff}=Uₛₕ-Uₑₗ 1

U_{diff} should preferably be small, i.e. close to or at 0, and remain stable, i.e. constant during use of the body electrode 100; 200, and in particular during the presence of the electrostatic field causing the disturbance. A constant and/or small U_{diff} value may result in an electrophysiological recording with at least a reduced amount of disturbances and/or a reduced amplitude of the disturbances. Constant shall be interpreted as to include small variations, such as a 10% variation, or a 5% variation or shall be interpreted as to include small variations in the potential such as a 10 mV variation, or a 6 mV variation or a 3 mV variation. The variation can be assessed during a time period, such as for example 1 min when the body electrode is subjected to electrostatic field disturbances. U_{diff} may remain at a value that enables that a signal-to-noise ratio between the recorded electrophysiological signal and a noise signal, e.g. induced by an electrostatic field, is not higher than 45 dB, or not higher than 40 dB.

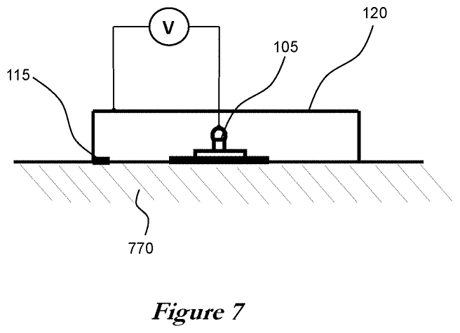

The potential difference between the transducer element 105; 205 and the skin contact element 115; 115′; 115″; 215, i.e. U_{diff}, may tested in a test equipment schematically illustrated in FIG. 7. Such a test equipment comprises an ion conducting material, e.g. 0.6% by mass NaCl in water, in the form of a solid material such as for example a hydrogel forming an ion conductive volume 770, which may be referred to as a phantom, which is used as a model of human or animal skin. The hydrogel, or the ion conductive volume 770, should have a substantially flat surface on which the body electrode 100; 200 is placed so that the skin contact element 115; 115′; 115″; 215 is in contact with the ion conductive volume 770. When the body electrode 100; 200 is placed on the ion conducting volume 770 the potential difference, U_{diff}, between the transducer element 105 and the electrical shield layer 113 of the layered shield structure 120 can be measured using a voltmeter or oscilloscope for a time period in the order of 1 minute. In order for the body electrode 100 to be able to conduct an electrophysiological measurement with reduced or minimized disturbances the measured U_{diff} should be lower than 50 mV, or lower than 30 mV, or lower than 20 mV, or lower than 15 mV, or lower than 10 mV, or lower than 7 mV, or lower than 5 mV during the measurement described above. The U_{diff} value, may depend on the material composition of the transducer element 105, the material composition of the skin contact element 115, and the material composition of the layered shield structure 120 as is further discussed below. The measured U_{diff} measured as a DC voltage should not vary more than 10% variation, or a 5% variation for the values outlined above for a measurement period of 1 minute. Preferably, the measured U_{diff} measured as a DC voltage should not vary more than 10 mV, or 6 mV or not more than 3 mV for a measurement period of 1 minute.

The measurement instrument probes may preferably be selected depending on/in respect to the material under test specifically certain shielding material which is appreciated by a person skilled in the art. For example, regular/standard stainless steel measuring instrument probes may typically be selected for electronic conducting material, e.g. metals. For ionic conducting materials a reference Ag/AgCl electrode can be used as probes, stainless steel probes or other probes having insufficient electrical/electrochemical stability may introduce measurement errors. For electron conducting material being fragile i.e break upon mechanical impact from regular stainless steel probes or materials in other ways inappropriate to use regular stainless steel probes as the ones described above, such as for example a thin layer structure with a layer thickness of 10-50 μm, it may be beneficial to use a probe comprising of a stable Ag/AgCl transducer element and an electrolyte medium comprised of Cl—ions, e.g. NaCl.

For measurement probes having a stable electrochemical potential this should be deduced from the measured U_{diff}, i.e if an Ag/AgCl electrode has been used with an electrochemical potential of 15 mV, 15 mV should be deduced from the measured U_{diff}.

A constant Udff may be achieved by a constant and/or stable Uₛₕ and/or U_{diff} value. Such a stable value for Uₑₗ may be achieved by a transducer element 105; 205 comprising a metal, e.g. Ag, coated with a metal salt with a low aqueous solubility, e.g. AgCl, which hereinafter will be referred to as a standard type of transducer element. A stable Uₛₕ value may be achieved by a skin contact element 115; 115′; 115″; 215 comprising a metal, e.g. Ag, coated with a metal salt, e.g. AgCl and an ion conducting layer 114; 214; comprising a salt, e.g a water-soluble salt such as NaCl, KCl or CaCl₂.

According to one embodiment wherein the transducer element is of standard type. The skin contact element comprises silver metal Ag with a conductive medium comprising a gel or hydrogel with at least 0.6% by mass of a chloride salt i.e. NaCl, KCl and CaCl₂.

According to one embodiment wherein the transducer element is of standard type and the electrolyte medium comprises a gel or hydrogel with at least 0.6% by mass of a chloride salt i.e. NaCl, KCl or CaCl₂, the electrically conductive layer of the skin contact element comprises silver (Ag).

According to one embodiment wherein the transducer element is of standard type. The skin contact element comprises silver Ag coated with AgCl with a conductive medium comprising a gel or hydrogel with at least 0.6% by mass of a chloride salt i.e. NaCl, KCl and CaCl₂.

According to one embodiment wherein the transducer element is of standard type and the conductive layer of the skin contact element comprises carbon coated with an acrylic polymer matrix wherein the polymer matrix (ion conductive layer) may serve as both electric conductive and ion conductive.

According to one embodiment wherein the transducer element is of standard type and the skin contact element comprises carbon coated with an electric conducting acrylic polymer matrix and a water-soluble salt with at least 0.6% by mass of a chloride salt i.e. NaCl, KCl and CaCl contained in the polymer matrix.

According to one embodiment wherein the transducer element is of standard type, the electrolyte medium comprises a gel or liquid with at least 0.6% by mass of a chloride salt i.e. NaCl, KCl and CaCl₂. and the skin contact element comprises an ionic conductive layer containing a gel or liquid with at least 0.6% by mass of a chloride salt i.e. NaCl, KCl and CaCl₂.

According to a further aspect of the above embodiment wherein the polymer matrix is an adhesive adhering to a substantially flat surface which could be a skin surface.

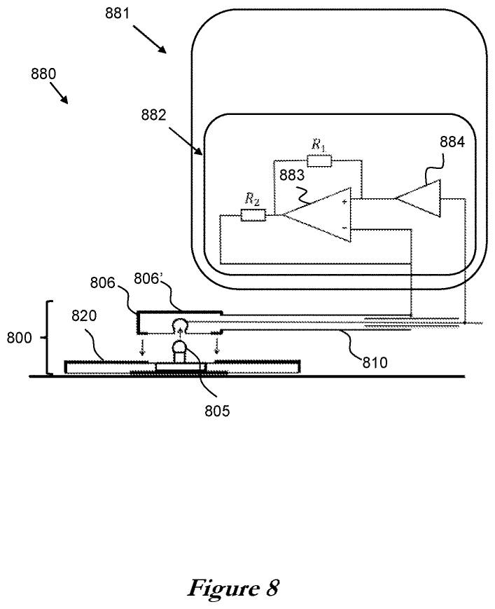

According to one aspect of the invention a measurement system 880, schematically illustrated in FIG. 8 is provided. The measurement system 880 comprises one or more body electrodes 800 each connected to a device for electrophysiological measuring 881 via a connector 806 and a lead 810 and a device for electrophysiological measuring 881. The device for electrophysiological measuring 881 comprise an electric potential equalization circuit 882 which comprises a measuring part 884 for measuring the electric potential of the transducer element 805, and a feedback part 883 that, based on measurement from the measuring part 888, provides the layer shield structure 820 with an electric potential such as to actively minimize the electric potential difference between the transducer element 805 and the layered shield structure 820 e.g. actively setting and/or keeping a voltage corresponding to U_{diff} at a predetermined value, typically close to 0 V. In an embodiment the feedback part 883 that, based on measurement from the measuring part 888, provides a connector shield 806′ with an electric potential. In such embodiment the connector shield 806′ is connected to the layered shield structure 820 so that the electrical layers of the respective shields are in electrical contact.

According to one embodiment the device for electrophysiological measuring 881 comprises an electric potential equalization circuit 882 and is used with one or more body electrodes 800 which are shielded by layered shield structure 820 comprising an electrical and an electrically dissipative layer but not provided with a skin contact element.

According to one embodiment the device for electrophysiological measuring 881 comprises an electric potential equalization circuit 882 and is used with the body electrodes 100, 200, 400 described with reference to FIGS. 1-5, which body electrodes comprises a skin contact element 115; 215. In this embodiment the matched potential characteristics of the transducer elements 105; 205 and the skin contact elements 115; 215 may be referred to as a passive potential equalization and the potential equalization provided by the device for electrophysiological measuring 881 comprising an electric potential equalization circuit as an active. It might be advantageous to combine the passive and active potential equalization, if for example a small potential difference exist also after the careful design of the body electrode and to compensate for time varying potential differences or differences that can be described to varying use conditions.

The measurement part 884 may have input characteristics typically required for an electrophysiological differential amplifier, such as used in equipment for electrophysiological signal recording and/or monitoring. The measurement part 884 preferably has a gain equal to 1 and is preferably an amplifier of a so called voltage follower type, i.e. with gain equal to one, having suitable input characteristic such as input impedance, typically in the order of 10-100 MOhm, and an input off-set current, typically in the order of 5-50 nA. The feedback part 883 may be based on an operational amplifier. The electric potential equalization circuitry operates to actively bring the electrical potential difference between the layered shield structure 820 and/or connector shield 806′, and the transducer element 805 down and towards a zero level.

The measurement system 800 system according to the invention is via the electric potential equalization circuitry configured to record a signal received by a transducer element 805 of a body electrode 800 and perform process the signal according to the continuous main steps of:
a) measure the electrical potential of the transducer element 805; and
b) provide an electric potential to the electrically conducting shield layer of the layered shield structure 820 or the connector shield 806′ wherein the electric potential is based on the measured electric potential in step a.

The method is capable of delivering a U_{diff} that is should be lower than 30 mV, or lower than 20 mV, or lower than 15 mV, or lower than 10 mV, or lower than 7 mV, or lower than 5 mV.

In one embodiment an aggregate arrangement 517 comprising an indifferent electrode 521 used as a skin contact element may be provided with a device for electrophysiological measuring 881. In such embodiment the measurement part 884 measures the electric potential via the indifferent electrode and the feedback part 883 provides the connector shields 806′ of the other body electrodes 500 with an electric potential, based on the measured electric potential from the indifferent electrode.

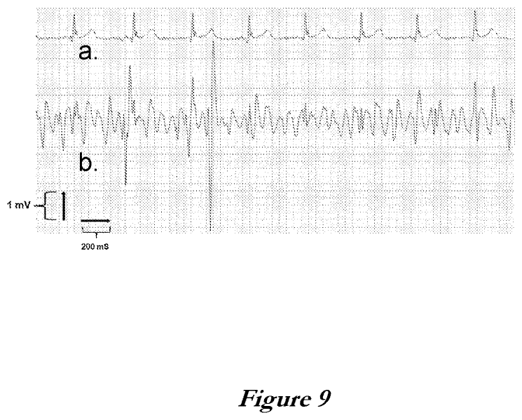

FIG. 9 show two electrocardiogram recordings using bipolar body electrodes on a human at close locations using electrocardiogram amplifiers with the same amplifications. Each spike shown on the curves represents disturbances in the signals obtained for the same type of electrostatic disturbance. Curve a) shows a recording using a body electrode 100 according to the invention and curve b) shows a recording using a body electrode according to the prior art. The measurement units for both electrocardiograms are the same and are displayed with a vertical arrow representing 1 mV and with a horizontal arrow representing 200 ms.

All embodiments, variants and examples may be combined with each other unless stated otherwise.

Implementation example:

For an ECG recording of diagnostic quality, it is desirable with a reduction of electrostatic noise in the order of a 100 times reduction in amplitude. In line with the above described embodiment a ECG body electrode was produced and tested. The outer shape of a body electrode used was a square with a 33 mm side. A shield structure was a circular electrode shield with a diameter of 25 mm. The body surface contacting electrode (transducer element) area was 18 mm in diameter, the transducer element/electrode conductive medium interface was of silver/silver chloride with a conductive hydrogel with at least 2% by mass sodium chloride as the electrode conductive medium. There was a collar surrounding the electrically conducting electrode portion and with a thickness of 1.5 mm. The skin contact element was a 0.3 mm diameter single strand wire 30 mm long made of silver which during use was in contact with the body surface. A connector shield overlapped a cable, i.e. lead, shield with at least 5 mm. The cable shield was connected electrically to a shielding circuit of an amplifier. The body electrode shield and the connector shield were not electrically connected with the cable shield. There were no holes or openings in the shield structure, formed by the electrode shield and connector shield, larger than a rectangle with sides 1.5 mm×0.5 mm. The device/body surface impedance was approximately 50 kOhm, for frequency range 6-100 Hz. The air room temperature was 22 degree Celsius and the relative humidity was 50%. The potential difference between the shield structure and the transducer element, i.e. U_{diff}, was measured to maximum 20 mV DC with a change, peak to peak, of maximum 2 mV per minute. With the same device described but without any electric potential equalization arrangement, i.e. said body surface contact element, and without the shield structure, disturbances on the electrophysiological signal of up to 2 mV amplitude is observed. With devices according to embodiments herein, electrostatic disturbance impact was below 0.01 mV in amplitude.

1. A body electrode for electrophysiological signal monitoring, the body electrode during used arranged to be attached to the skin of a subject, wherein the body electrode comprises:
a collar with at least one through opening;
a transducer element at least partly arranged within the through-opening of the collar;
a skin facing surface arranged to, during use, be in contact with the skin and a free surface opposite of the skin facing surface;
a connector in electrical contact with the transducer element, the connector arranged on the free surface; and
an electrically conducting compartment formed by the wall of the through-opening of the collar and comprising the transducer element, the electrically conducting compartment during use comprising an electrolyte medium;
wherein the body electrode comprises a layered shield structure arranged on the free surface of the body electrode, wherein the layered shield structure comprises at least an electrically conducting layer and an electrostatic dissipative layer arranged on the surface of the electrical conducting layer, and wherein the layered shield structure covers covcring at least the electrically conducting compartment, the connector and the transducer element, and
wherein the body electrode comprises a skin contact element in electrical contact with the layered shield structure, the skin contact element comprising at least an ion conducting layer comprising ion conducting material and an electrically conducting layer comprising electrically conducting material, wherein the materials of the layers of the skin contact element are selected so that the electrical potential of the skin contact element matches the electrical potential of the transducer element, and wherein the skin contact element is arranged to during use be in contact with the skin.

2. The body electrode according to claim 1, wherein the layered shield structure of the body electrode further comprises an ion-conducting layer, and
wherein the ion-conducting layer of the skin contact element is arranged to be connected with the ion-conducting layer of the layered shield structure.

3. The body electrode according to any of the prcccding claims claim 1, wherein the layered shield structure further comprises an ionic conducting layer arranged below the electrically conducting layer, and
wherein the layered shield structure covers at least the electrically conducting compartment, the connector and the transducer element.

4. The body electrode according to claim 2, wherein the skin contact element is formed by a portion of the layered shield structure arranged to extend to the skin facing surface.

5. The body electrode according to claim 1, wherein the skin contact element is a wire-like structure extending from the electrical shield layer on the outer peripheral of the collar and over at least a portion of the skin facing surface of the body electrode.

6. The body electrode according to claim 1, wherein the layered shield structure further comprises at least one flap arranged to during use spatially overlap with a lead shield.

7. The body electrode according to claim 1, wherein at least one of the materials of the skin contact element; differs from the materials of the transducer element.

8. The body electrode according to claim 1, wherein the potential difference U_{diff}, between the transducer element and the electrically conducting layer is lower than 50 mV, preferably lower than 30 mV, and even more preferably lower than 10 mV.

9. The body electrode according to claim 1, wherein the ion conducting layer comprises a polymer matrix and a water-soluble salt.

10. The body electrode according to claim 8, wherein the polymer matrix comprises a hydrogel.

11. The body electrode according to claim 1, wherein the electrically conducting layer comprises a carbon-based polymer material and the ion conducting layer comprises an acrylic material.

12. The body electrode according to claim 1, wherein the ion conducting layer is adhesive.

13. The body electrode according to claim 1, wherein the ion conducting layer is an adhesive layer that is arranged to, during use, also fasten the skin contact element to the skin.

14. The body electrode according to claim 1, wherein the transducer element comprises Ag/AgCl.

15. The body electrode according to claim 13, wherein the skin contact element comprises Ag and a gel or hydrogel with at least 0.6% by mass of a chloride salt.

16. The body electrode according to claim 13, wherein the skin contact element comprises Ag coated with AgCl and a gel or hydrogel with at least 0.6% by mass of a chloride salt.

17. The body electrode according to claim 13, wherein the skin contact element comprises a carbon layer coated with an acrylic polymer matrix, and thereby the carbon layer coated with an acrylic polymer matrix function as both the electrically conducting layer and the ion conductive layer.

18. The body electrode according to claim 1, wherein the electrically conducting layer of the body electrode and/or the electrically conducting layer of the skin contact element comprises an ion carrier material comprising an ion solution and thereby functions as both the electrically conducting layer and the ion conducting layer.

19. A body electrode and lead connector assembly, comprising:
the body electrode according to claim 1; and
a lead connector,
wherein the lead connector is arranged to be mounted to the connector of the body electrode, and the layered shield structure of the body electrode is provided with a through opening through which the connector is arranged to extend, and
wherein the lead connector is provided with a layered shield structure comprising an electrically conducting layer and an electrostatic dissipative layer and in the mounted position the conducting layer of the body electrode is in electrical contact with the electrically conducting layer of the lead connector, and the electrostatic dissipative layer of the body electrode is arranged to be in physical contact with the electrostatic dissipative layer of the lead connector.

20. A body electrode arrangement, comprising:
at least one body electrode;
an indifferent body electrode; and
a hub device,
wherein the indifferent body electrode the body electrode according to claim 1, and
wherein in the body electrode arrangement each of the body electrodes comprises a transducer element that during use is connected to a measuring device via an signal conductor, and
wherein each of the body electrodes comprises:
a layered shield structure comprising an electrically conducting layer and an electrically dissipative layer, and
an isolated lead passing the hub device and electrically connecting the electrically conducting layer of the each of the body electrodes, and
wherein the indifferent body electrode provides a skin contact element that is arranged to be in electrical contact with the isolated lead .

21. A body electrode arrangement, comprising:
at least one body electrode;
an indifferent body electrode; and
a hub device,
wherein each of the body electrodes comprises a transducer element that during use is connected to a measuring device via an signal conductor,
wherein, each of the body electrodes comprises a layered shield structure comprising an electrically conducting layer and an electrically dissipative layer; an isolated lead passing the hub device and electrically connecting the electrically conducting layer of the each of the body electrodes,
wherein the indifferent body electrode provides a skin contact element that is arranged to be in electrical contact with the isolated lead, and
wherein the indifferent body electrode comprises a transducer element utilized as a skin contact element and the isolated lead is connected to the transducer element of the indifferent electrode.

22. A measurement system comprising a measurement unit connected to a body electrode according to claim 1, further comprising means configured to:
measure an electric potential of the transducer element of the body electrodes; and
provide an electric potential to the electrically conducting layer of the layered shield structure,
wherein the electric potential is based on the measured electric potential, so that the potential difference, U_{diff}, between the layered shield structure and the transducer element is lower than 30 mV, preferably lower than 15 mV, and even more preferably lower than 10 mV.

23. A measurement system comprising a measurement unit connected to the body electrode arrangement according to claim 20, further comprising means configured to:
measure an electric potential via the indifferent electrode; and
provide the connector shields of the body electrodes with an electric potential, based on the measured electric potential from the indifferent electrode,
wherein the electric potential is based on the measured electric potential, so that the potential difference, U_{diff}, between the layered shield structure and the transducer element is lower than 30 mV, preferably lower than 15 mV, and even more preferably lower than 10 mV.
